(19) United States
(12) Patent Application Publication
(10) Pub. No.: US 2002/0006619 A1
(43) Pub. Date: Jan. 17, 2002

(54) Thermal cycler that allows two-dimension temperature gradients and hold time optimization

(76) Inventors: David Cohen, Dedham, MA (US); Michael J. Finney, San Francisco, CA (US); Michael Mortillaro, Lunenburg, MA (US)

Correspondence Address: MINTZ, LEVIN, COHN, FERRIS,, GLOVSKY and POPEO, P.C, One Financial Center, Boston, MA, 02111, US

(21) Appl. No.: 09/791,367

(22) Filed: Feb. 23, 2001

Related U.S. Application Data
(60) Provisional application No. 60/184,480, filed on Feb. 23, 2000.

(51) Int. Cl. C12M 1/34; H05B 3/68; C12Q 1/68

(52) U.S. Cl. 435/6; 435/287.2; 219/447.1

(57) ABSTRACT

A thermal cycler for use in thermal cycling procedures, and more specifically to a thermal cycler and a method for using same which permits the creation of temperature gradients in the thermal cycler in either of two dimensions and which permits optimization of the hold time of a given step in the thermal cycling procedure

### FIELD OF THE INVENTION

The present invention is directed to a thermal cycler for use in thermal cycling procedures, and more specifically to a thermal cycler and a method for using same which permits the creation of temperature gradients in the thermal cycler in at least two dimensions independently and which permits optimization of the hold time of a given step in the thermal cycling procedure.

### BACKGROUND OF THE INVENTION

Molecular biology thermal cyclers are instruments adapted for performing any of several types of reaction, the most common being polymerase chain reaction (“PCR”) with a thermostable polymerase (Mullis et al., U.S. Pat. Nos. 4,683,195; 4,683,202; 4,965,188) and thermal cycle DNA sequencing (Innis et al., U.S. Pat. No. 5,075,216). There has long been an interest in finding quick and easy ways to optimize these protocols. Temperature optimizations have been commonly performed in a temperature-gradient thermal cycler (Danssaert et al., U.S. Pat. Nos. 5,525,300; 5,779,981).

Several in vitro nucleic acid amplification reactions require that a reaction mixture be thermally cycled. Examples include the Polymerase Chain Reaction, thermal cycle DNA sequencing, and the Ligase Chain reaction. Typically a reaction mixture contains a nucleic acid template, various reagents, enzymes, one or more oligonucleotides and possibly fluorescent or radioactive markers. If a given reaction is to be used frequently, it is worthwhile to optimize the parameters of the reaction to ensure maximum product yield, shortest reaction time, and lowest reagent costs. These parameters include chemical concentrations in the solution, the hold temperatures within the thermal cycling protocol, and the hold times for each temperature step. Varying the solution from sample to sample and analyzing the results can optimize chemical concentrations.

Using a temperature-gradient-enabled thermal cycler allows easy optimization of hold temperatures. A PCR or thermal cycle sequencing reaction consists typically of two or three temperature hold steps interspersed with rapid temperature changes or “ramps”. The steps include: “denaturation” which allows strand separation; “annealing” which allows one or more oligonucleotide primers to pair with the template; and “extension” which is optimized for the synthetic activity of the polymerase enzyme. The annealing and extension steps are frequently combined into a single annealing/extension step.

A thermal cycler normally has a metal block with recesses formed in a top surface that holds samples in plastic vessels in an X-Y grid or other pattern such as a rectangular or hexagonal grid, and subjects them all to heating steps at a series of temperatures, as uniformly as possible, at the direction of a programmed controller that may include a computer central processing unit or other suitable microcontroller. A one-dimensional temperature gradient thermal cycler is one which is capable of producing a temperature gradient in a preferred direction (e.g., the X direction). Thus, a series of samples arrayed in the X direction can be subjected to a series of heating steps, where the temperatures are identical for some of the heating steps, but cover a range of temperatures for a particular step (or a repeated step in a repeated subset). This segregates the test samples into distinct temperature regions for that step that correspond to columns of samples in the Y direction. Because biochemical processes, such as nucleic acid primer annealing, vary significantly with temperature over a range of several degrees, a temperature gradient must cover a range of at least two degrees in order for results to be useful. By analyzing the reaction product from samples in more than one column for some measure of quality, it is possible to closely approximate, after only one experiment, the temperature for that heating step that optimizes product quality. Thus the optimum temperature can be determined for a given step.

However, currently available instruments only allow one temperature step to be independently optimized in a given experiment. Some instruments, such as those manufactured by Stratagene of La Jolla, Calif., and disclosed in U.S. Pat. No. 5,525,300 and 5,779,981, have separate metal blocks for each temperature, only one of which is capable of generating a temperature gradient. Other instruments, such as those manufactured by MJ Research, Waltham Massachusetts, Eppendorf Scientific, Inc. of Westbury, New York, and Biometra of Gottingen, Germany, change the temperature of a single metal block, and can form a single-dimension thermal gradient in that block. While it would be possible to form temperature gradients at more than one step using the latter technology, the two temperature gradients would be aligned along the same axis, and thus the results would be confounded.

In addition to optimizing temperatures, it is also useful to optimize the times for which the temperatures are held at those temperatures at those temperatures (so called “hold times”). For instance, long hold times at an “extension” step may be necessary to synthesize long product molecules; hold times that are too short decrease product yield. However, hold times that are longer than necessary waste resources and limit the throughput possible with a given number of instruments. Longer than necessary hold times can also contribute to the generation of unwanted products in PCR or cycle sequencing reactions, resulting in background or “smears” on gels. In the “denaturation” step, long hold times result in progressive irreversible inactivation of the synthetic enzyme. Thus, more enzyme is needed per reaction to compensate for expected enzyme loss. As enzymes account for a large percentage of the cost of a reaction, minimizing the amount used per sample can lead to considerable cost savings. However, “denaturation” hold times that are too short may not allow the entire sample to reach the melting temperature, decreasing reaction yield. It is therefore highly beneficial to allow protocol designers an easy method of optimizing a temperature hold time by means of a single experiment. There is currently no fast, easy way to determine optimum hold times.

### SUMMARY OF THE INVENTION

A thermal cycler designed for rapid optimization is presented here. In one embodiment such a cycler can create a temperature gradient in either of two dimensions (referred to as “2D Grad” or “2D Gradient”) across the temperature-controlled element commonly referred to as a “block,” thus allowing a user to optimize the temperature of two cycling steps of a protocol with a single experiment. Other embodiments allow thermal gradients to be established in three or more directions. Another embodiment of the present invention is directed to a method for the use of the thermal cycler described above for optimizing temperatures in cycling protocols. Finally, there is described a method for using a gradient-enabled thermal cycler to optimize the hold time of a certain temperature steps for use with PCR or thermal cycle DNA sequencing.

The preferred embodiment provides a thermal cycler for providing a two-dimensional temperature gradient wherein a second temperature gradient, perpendicular to the first gradient, is formed at a different step from the first gradient. The thermal cycler controls the temperature of a rectangular metal block in which recesses for receiving samples or sample-holding containers are formed into an upper surface, forming an X-Y grid of sample recesses. The metal block is not, however, limited to a rectangular configuration. Other exemplary blocks include those having a hexagonal configuration.

If the first gradient is formed in the X direction, the second gradient is formed in the Y direction, dividing the test samples into temperature regions corresponding to rows and columns of wells. For any given row, the samples are exposed to the same temperature conditions throughout the entire protocol, except for when the X gradient is formed. Similarly, for any given column, the samples are exposed to the same temperature conditions throughout the entire protocol except for when the Y gradient is formed. This allows simultaneous temperature optimization of a second step in the protocol without impacting the results of the optimization of the first step. One sample from each of a plurality of columns is still analyzed to determine the optimum temperature of the first step, and one sample from each of a plurality of rows is used to determine the optimum temperature for the second step. In certain cases, it is expected that the optimum temperatures will not be independent of each other. In such cases, samples derived from a grid consisting of a plurality of rows and a plurality of columns must be tested in order to determine an optimum protocol consisting of a co-optimized pair of temperatures for the two steps under investigation. In embodiments in which the block is not rectangular, e.g. hexagonal, the angles between the first direction of the temperature gradient and the second direction of the temperature gradient is at least 30° but less than 150°.

The present invention also provides a thermal cycler and a method for its use, which is suitable for controlling the hold time of a given step differently in different parts of the thermal cycler block.

In a PCR or cycle sequencing temperature cycle, there is only one point at which no reaction of any practical consequence is occurring. During the denaturation step, strands are separating and enzyme is becoming inactivated; while ramping from denaturation to annealing, the separated strands are reannealing, a reaction that competes with primer annealing. During the annealing step, primers begin to be extended. During the extension step, extension of the primers continues. However, after the extension step, when the temperature of the sample has reached about 85° C., enzymatic activity has virtually ceased, while the temperature is too low to begin the separation of strands or to inactivate the enzyme.

A time gradient may be performed for either the denaturation step or the step immediately preceding it (extension or annealing/extension). The time gradient is executed by creating a temperature gradient in between the two steps, such that some of the samples are in the temperature range of one of the hold steps, while other samples are in an inactive temperature range.

### DETAILED DESCRIPTION OF THE DRAWINGS

The invention will be better understood by reference to the appended figures of which:

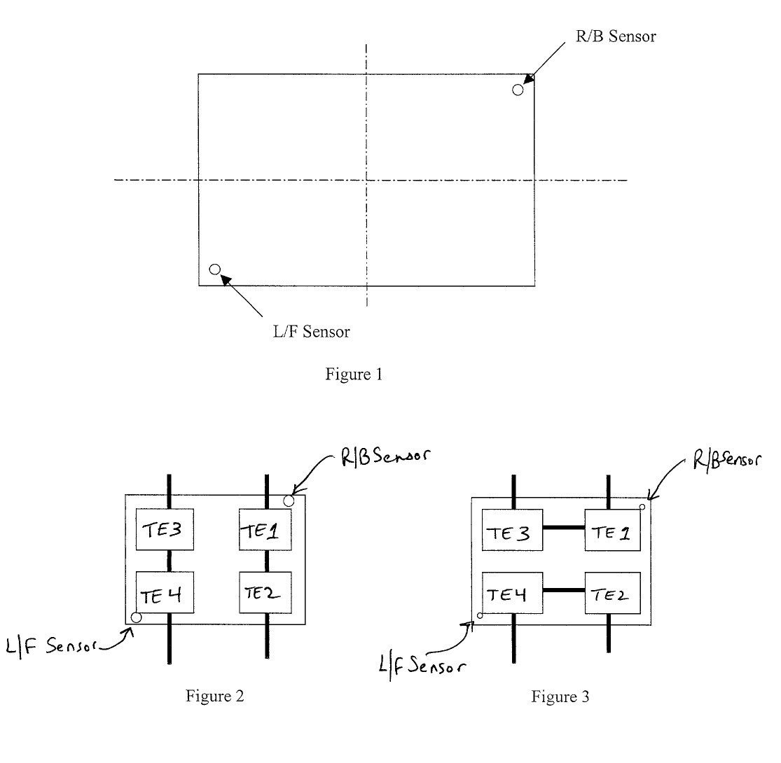

FIG. 1 is a block diagram which illustrates the distribution of temperature control zones and sensors on a temperature block in accordance with one embodiment of the present invention;

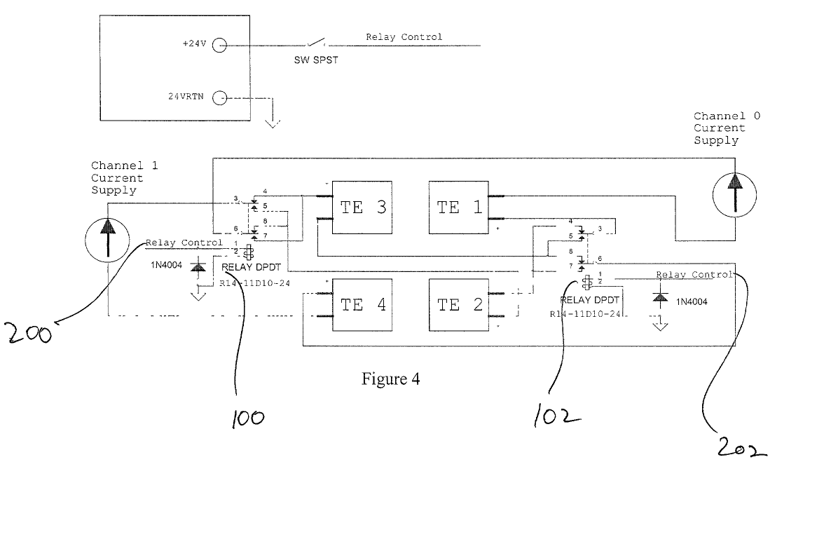

FIG. 2 is a block diagram illustrating the temperature control zone configuration used to create a Left/Right gradient in a temperature block in accordance with one embodiment of the present invention;

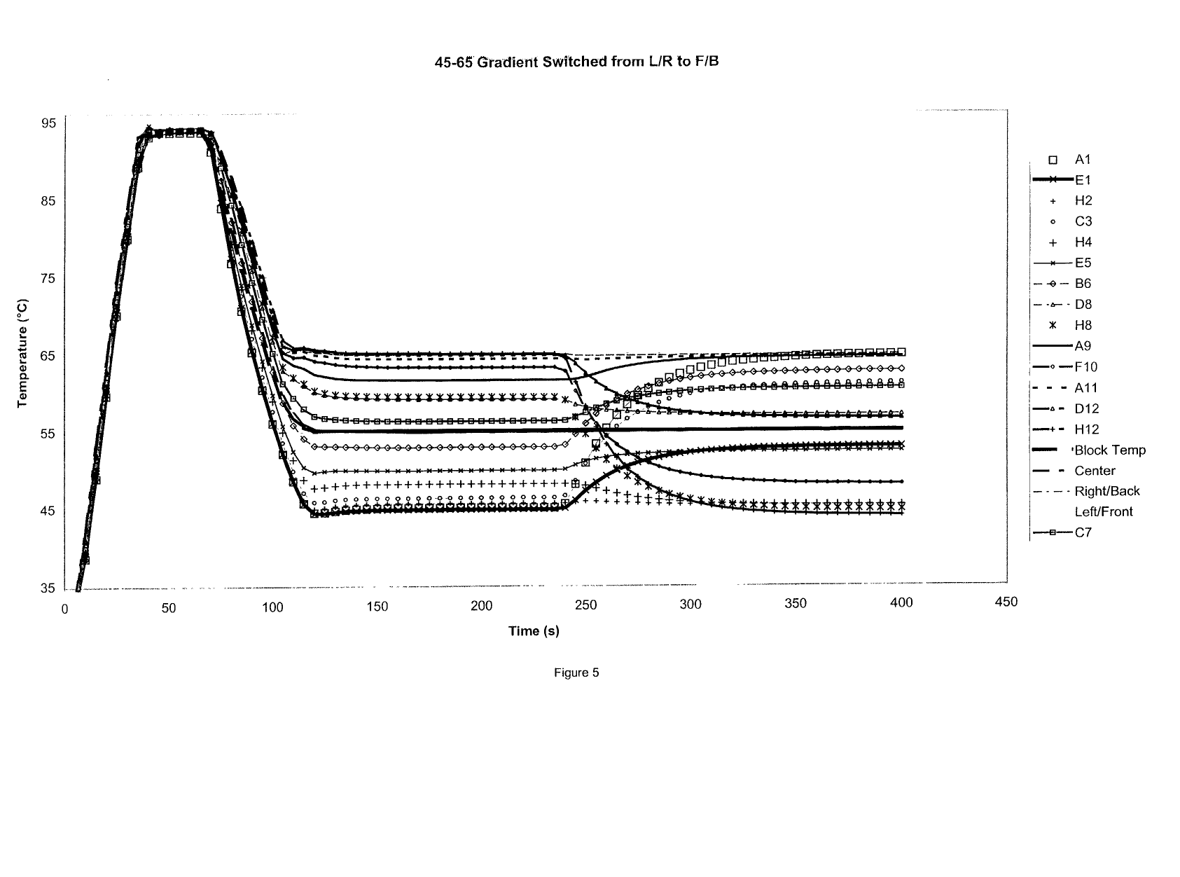

FIG. 3 is a block diagram illustrating the temperature control zone configuration used to create a Front/Back gradient in a temperature block in accordance with one embodiment of the present invention;

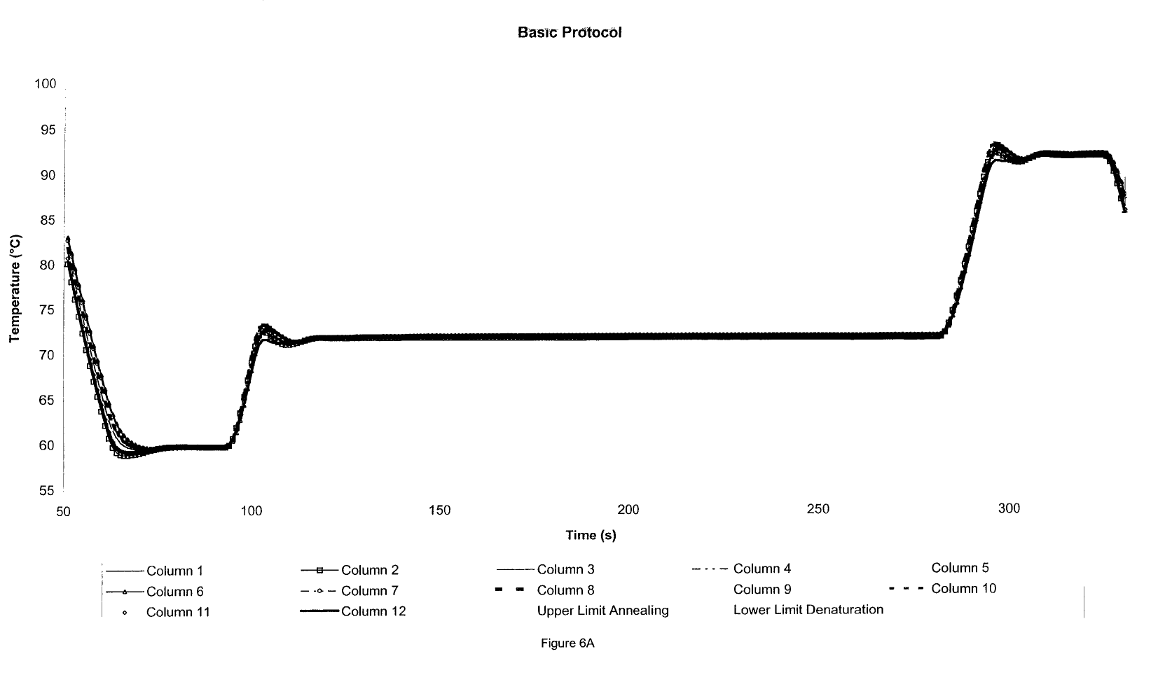

FIG. 4 is a circuit diagram which illustrates the controlling circuitry for producing a two-dimension temperature gradient in a temperature block in accordance with the present invention.

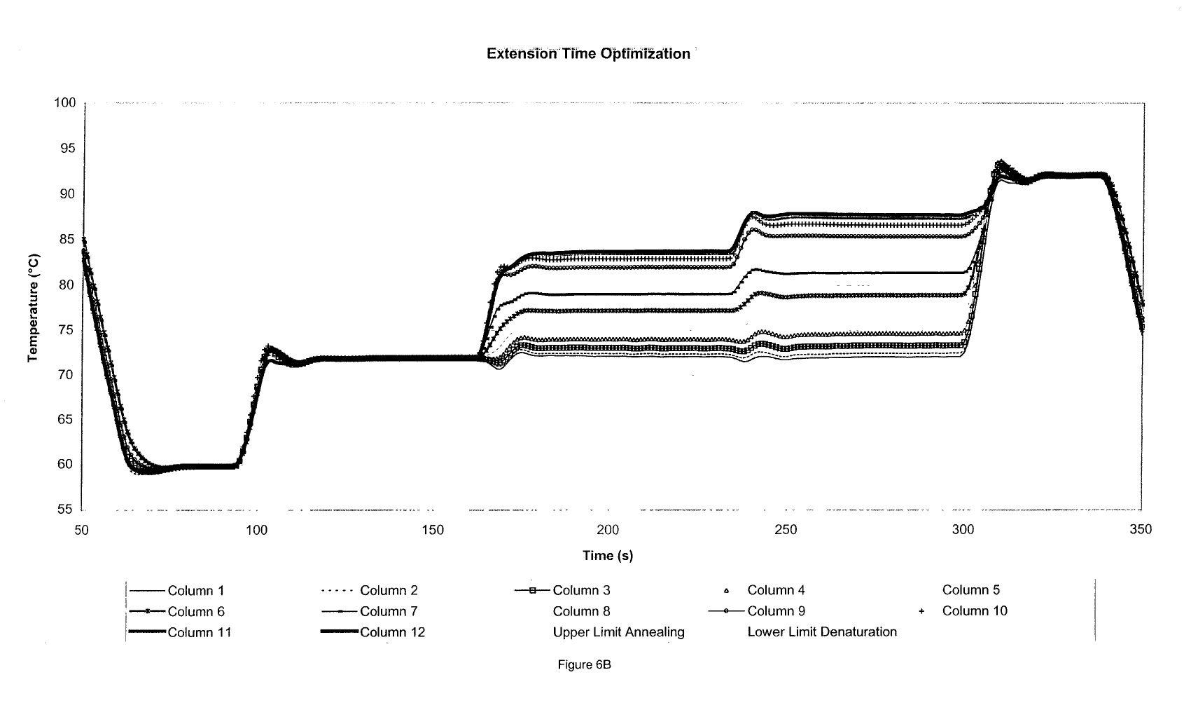

FIG. 5 is a graph which illustrates a gradient shift from one dimension (Left/Right) to another dimension (Front/Back) in accordance with the present invention;

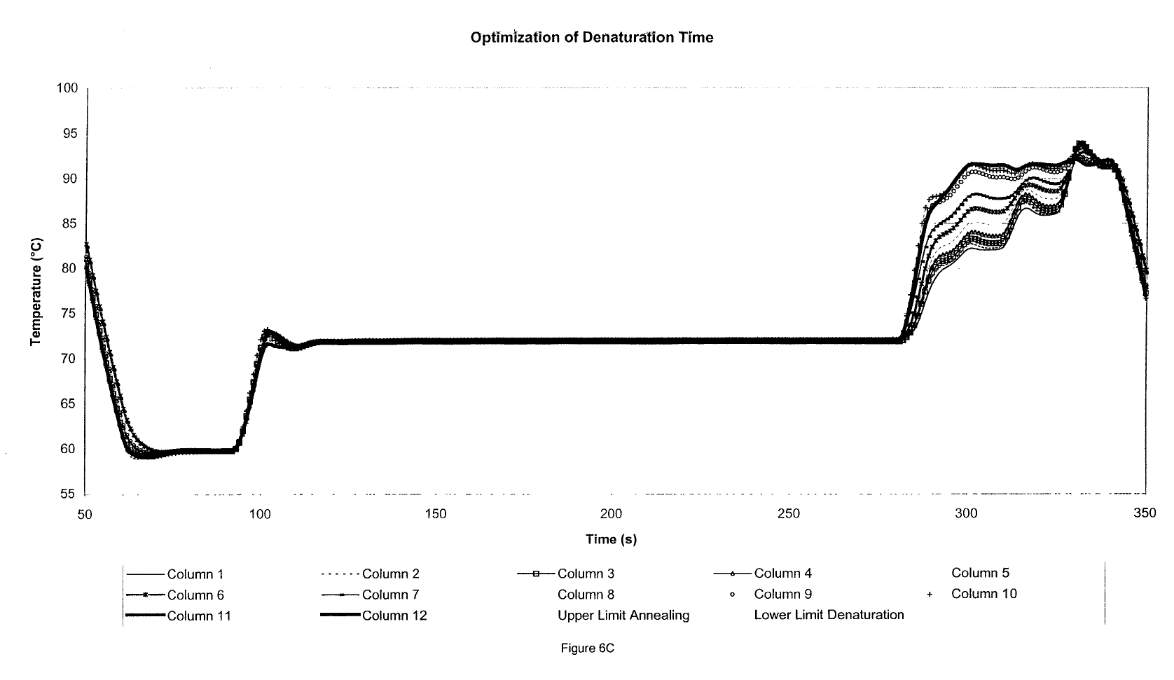

FIG. 6A is a graph which illustrates the operation of a basic protocol without utilizing hold time optimization;

FIG. 6B is a graph which illustrates the basic protocol in FIG. 6A modified to utilize hold time optimization of the extension step in accordance with one embodiment of the present invention;

FIG. 6C is a graph which illustrates the basic protocol in FIG. 6A modified to utilize hold time optimization of the denaturation step in accordance with one embodiment of the present invention;

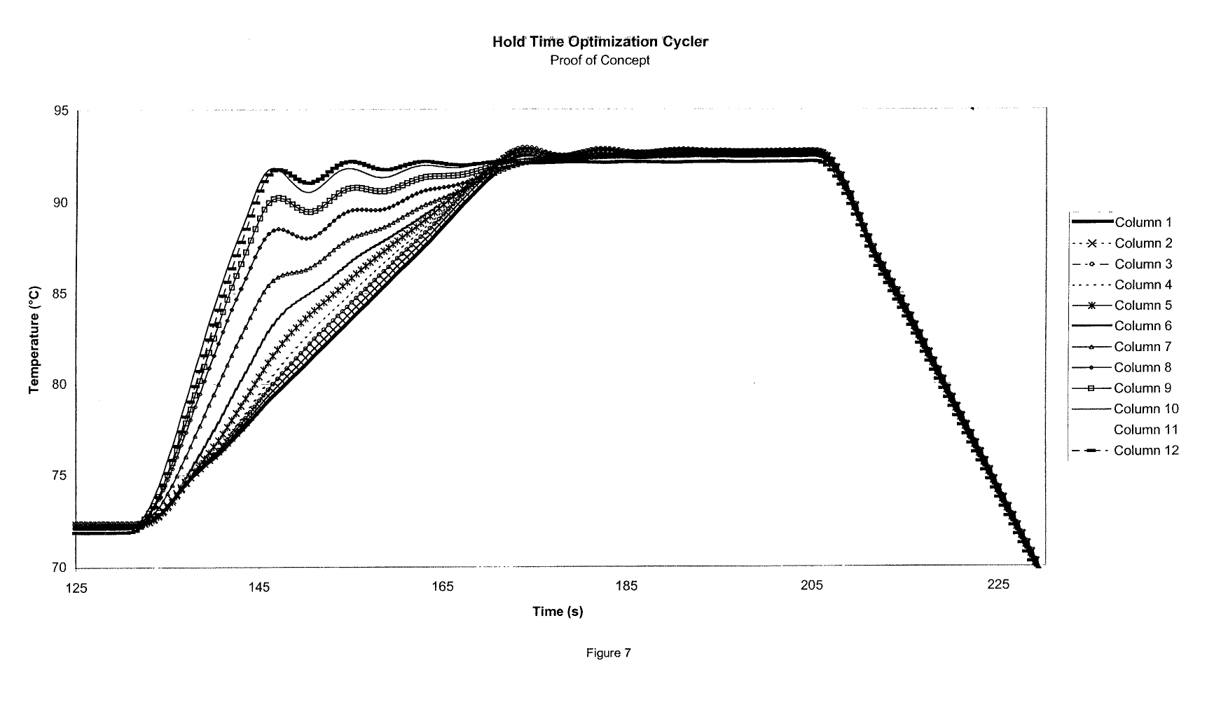

FIG. 7 is a graph which illustrates an alternative method of achieving hold time optimization in accordance with one embodiment of the present invention in which the temperature control zones are ramped at different rates to achieve the different hold times;

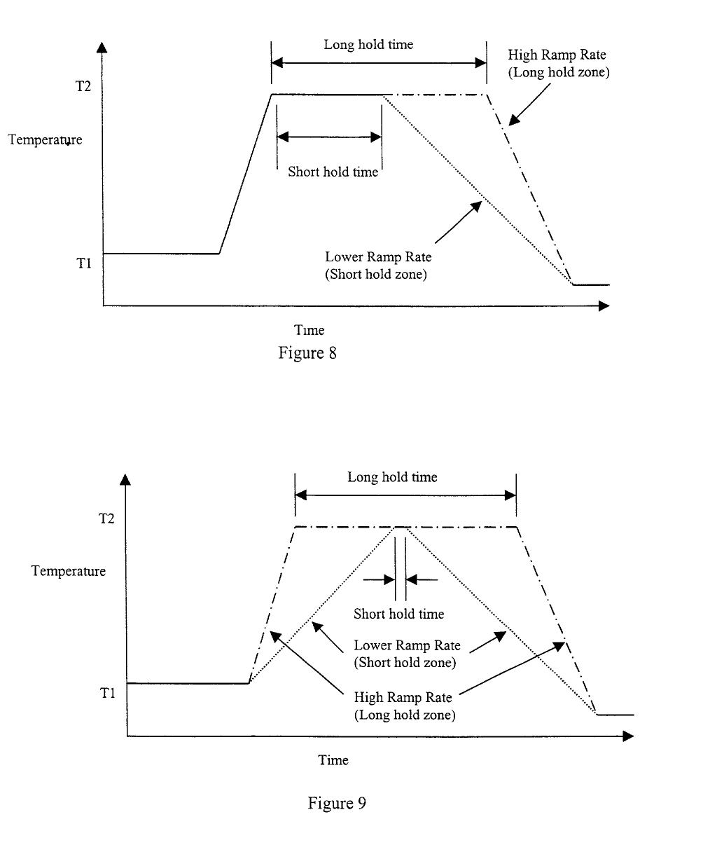

FIG. 8 is a graph which illustrates an alternative method of achieving hold time optimization shown in FIG. 7 which utilizes the trailing ramp instead of the leading ramp in order to optimize the hold time of the temperature step in accordance with one embodiment of the present invention; and

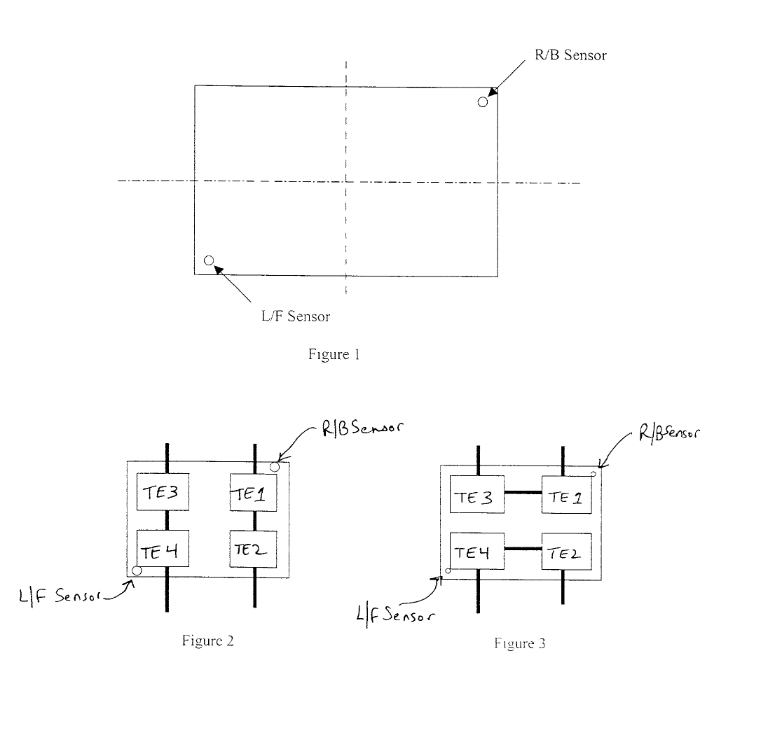

FIG. 9 is a graph which illustrates the control methods shown in FIGS. 7 and 8 combined so that the temperature control zones are ramped independently on both sides of the hold time portion of the cycle to achieve hold time optimization in accordance with one embodiment of the present invention.

### DETAILED DESCRIPTION OF THE INVENTION

The general design and construction of thermal cyclers is well known in the art. Common methods of controlling temperature in a thermal cycler include Peltier-effect thermoelectric heat pumps, electrical resistance heating elements (“Joule heaters”), fluid flow through channels in a metal block, either solely for cooling or both for heating and for cooling, using reservoirs of fluid at different temperatures; and tempered air impingement. Any of these techniques as well as others known in the art are capable of being used as temperature regulating elements to construct gradient-enabled thermal cyclers.

In order to form and maintain a temperature gradient across a thermally conductive sample block, it is necessary to control the heat flux into and out of the block differentially so that at least two distinct regions of temperature control are established. The location of the temperature control zones, the heat generating or heat removal capability of the temperature regulating elements, and the material composition and cross-sectional geometry of the block determines the maximum magnitude and shape of the achievable temperature gradient. In typical blocks that allow both gradient and non-gradient temperature control, the regions of temperature control are distributed symmetrically about an imaginary line that bisects the block into a left half and a right half. This allows the block to form a left to right temperature gradient for one or more steps of a temperature cycling protocol. The particular arrangement used in MJ Research PTC-200 series thermal cyclers is described in MJ Research publication #ssgr991209 (1999), entitled “The MJ Research Gradient Feature.”

In one embodiment of the present invention, the block is built such that the temperature control elements are distributed into right and left zones at some times during the protocol, and at other times the temperature control elements are distributed into front and back zones. Thus the instrument is capable of forming both left/right (“L/R”)and front/back (“F/B”) gradients as needed.

More specifically, the preferred embodiment employs Peltier-effect thermoelectric modules as part of the temperature control elements, supplemented with electrical resistance heating elements, such as Joule heaters. The sample block is divided into quadrants, as shown in FIG. 1. Temperature sensors are attached to the block at least in two diametrically opposed quadrants.

One thermoelectric module (“TE”) is used to control each quadrant. Because only two sensors are used to monitor the block temperature, the TEs need to be run as two circuits. As illustrated in FIG. 4, each circuit consists of two TEs in series. To form a left/right gradient, TEs 1 & 2 are driven together and monitored by the R/B sensor shown in FIG. 1. TEs 3 & 4 are also driven together and they are monitored by the L/F sensor as shown in FIG. 2.

To form a front/back gradient, TEs 1 & 3 are driven together and their temperatures monitored by the R/B sensor, and TEs 2 & 4 are driven together and their temperatures monitored by the L/F sensor as shown in FIG. 3. Each pair of TEs may be coordinately controlled by a single controller.

In the various embodiments of the present invention, other heat flux control mechanisms besides TEs can also be used. Examples include electrical resistance for heating and circulating fluid for cooling; or electrical resistance for heating and forced air for cooling. It is also possible to further subdivide the regions of control by adding more temperature sensors and heat flux control devices. Temperature sensors may be attached in all four quadrants. If four sensors instead of the two shown in FIG. 1 are being used, the four TEs can be driven independently to achieve the same results.

To demonstrate the manner in which the preferred embodiment functions in accordance with the present invention, a heat pump/control block module for a thermal cycler was modified to produce two-dimensional temperature gradients. The module was an MJ Research Rev 01 96v Alpha Unit serial number AL024887. It was modified by inserting mechanical relays such as the two relays 100 and 102 shown in FIG. 4, mounted outside the unit, into the circuit as shown in FIG. 4. This circuit, using techniques well known in the art, allows “line” switching of the circuit under control of the relay controls 200 and 202, between the configurations of FIG. 2 and FIG. 3 while the instrument is operating. The modified module was controlled by a standard MJ Research PTC-200 thermal cycler base. It was possible to “hot swap” the TEs at any time during a run by opening and closing the switch in the relay control circuit of FIG. 4. When switching is performed in the middle of a gradient step, the gradient smoothly shifts from one dimension to the other, as illustrated in FIG. 5. Distribution of the row temperatures in F/B gradient mode is similar in shape to distribution of column temperatures in L/R mode.

The thermal cycler of the present invention also provides for optimization of the hold time gradient. To optimize hold times, it is desirable to use a thermal cycler that creates a “hold time gradient” across the block. This means that for a given temperature step, the samples in one region of the block would experience a long hold time, while samples in other regions would experience a shorter hold times at the same temperature. This situation is difficult to achieve if the hold temperatures are precisely defined. However, as described hereinabove, in certain cases the precise temperature is less important than whether the temperature is within certain zones.

For the purposes of illustrating the various embodiments of the invention, temperatures are divided into three zones: the “active zone,” having temperatures below 82° C., where polymerases have significant activity; the “inactive zone,” having temperatures in the range from 82° C. to 88° C., where no significant reactions take place; and the “melting zone,” having temperatures above 88° C., where strand separation and irreversible enzyme inactivation can occur. These temperatures are approximations, and will vary in individual circumstances depending on factors such as enzyme type, monovalent and divalent cation concentrations, and product length.

In one embodiment, the following protocol, illustrated in FIG. 6A, is used as the starting point (all temperatures are in celsius):

| | | |
|---|---|---|
| 60° | 30 sec. | (annealing) |
| 72° | 180 sec | (extension) |
| 92° | 30 sec. | (denaturing) |

The extension time may be optimized, as illustrated in FIG. 6B, using the method of the invention. The cycler is programmed as follows:

| | | |
|---|---|---|
| 60° | 30 sec. | (annealing) |
| 72° | 60 sec | (extension) |
| 72-84° | 60 sec. | (gradient) |
| 72-88° | 60 sec. | (gradient) |
| 92° | 30 sec. | (denaturing) |

Thus, as the samples traverse from the extension step to the denaturation step, different samples will spend different amounts of time in the active zone. In this protocol, columns 1-3 will spend only 60 seconds in the active zone; columns 4-5 will spend 120 seconds in the active zone; and columns 6-12 will spend 180 seconds in the active zone. Thus, at least three times may be assayed to help discover the optimum time.

Similarly, the method of the invention may be used to optimize the denaturation step illustrated in FIG. 6C, as follows

| | | |
|---|---|---|
| 60° | 30 sec. | (annealing) |
| 72° | 180 sec | (extension) |
| 82-92° | 10 sec. | (gradient) |
| 86-92° | 10 sec. | (gradient) |
| 92° | 10 sec. | (denaturing) |

As the samples traverse from the extension step to the denaturation step, different samples will spend different amounts of time in the melting zone. In the protocol illustrated in FIG. 6C, columns 1-4 will spend 10 seconds in the melting zone; columns 5-7 will spend 20 seconds in the melting zone; and columns 8-12 will spend 30 seconds in the melting zone. Thus, at least three times may be assayed to help discover the optimum time.

In the case in which time at a specific temperature is determined to be more important than the amount of time spent in a temperature range, the thermal cycler can be altered to operate such that the zones of temperature control are ramped independently to target. By controlling the rate at which the zone is ramped, the time spent at the specific target can be specified. This can be demonstrated by a protocol in which the software in an existing MJ Research PTC200 DNA engine was modified to enable a 96v alpha be run with two independent control zones on the left and right sides. Resistance heater channels were turned off, and the TE power levels were adjusted to compensate. The results are illustrated in FIG. 7.

In this protocol, as illustrated in FIG. 7, the left-most column of the block (column 1) was held at 92.0° C. for thirty seconds, while the right most column of the block (column 12) was held at 92.0° C. for sixty seconds. Intermediate columns have no useful hold time optimization information for this particular hardware configuration, but if more control zones were to be added across the block, more useful time optimization information would be available corresponding to the added zones.

In an alternative method of creating the difference in hold times illustrated in FIG. 8, the ramp rates in the two control zones are controlled during the ramp down portion of the cycle, instead of in the ramp up portion. Alternate ramp rates may also be controlled in both the up ramp portion and down ramp portion of the cycle. The temperature profiles for this control scheme is shown in FIG. 9.

With regard to the protocols as illustrated in FIGS. 8 and 9, the solid temperature profile lines represent portions of the temperature cycle at which both control zones act to maintain a uniform temperature across the block. Dotted profile lines show the control path set for the short hold time zone of the block, and dot-dash profile lines show the control path set for the long hold time zone of the block. Note that once again these representations apply for cyclers that have only two control zones. Additional control zones would add the ability to set additional hold times in an experiment.

From the foregoing detailed description of the specific embodiments of the invention, it should be apparent that a unique thermal cycler and method of using said thermal cycler in thermal cycling procedures has been described. Although particular embodiments have been disclosed herein in detail, this has been done by way of example for purposes of illustration only, and is not intended to be limiting with respect to the scope of the appended claims that follow, and it will be understood that various omissions, substitutions and changes in the form and details of the disclosed invention maybe made by those skilled in the art without departing from the spirit of the invention. In particular, it is contemplated by the inventor that various substitutions, alterations, and modifications may be made to the invention without departing from the spirit and scope of the invention as defined by the claims.

What is claimed is:

1. A thermal cycling instrument comprising:
a metal block with recesses formed into a first surface for receiving samples;
at least three independently-controllable temperature regulating elements in thermal communication with said metal block; and
a programmable controller capable of controlling the temperature regulating elements independently, wherein, independent control of the temperature regulating elements is sufficient to achieve a temperature gradient of at least 2 degrees C. in either a first direction or a second direction, and wherein said first direction and said second direction are substantially parallel to said first surface and the angle between said first direction and said second direction is at least about 30 degrees but less than about 150 degrees.

2. A thermal cycling instrument according to claim 1, wherein the first surface of the metal block is essentially rectangular, and at least four temperature regulating elements are in respective thermal communication with the four quadrants of said metal block.

3. A thermal cycling instrument according to claim 2, wherein the at least four temperature regulating elements are controlled by arranging them as 2 adjacent pairs, wherein each pair is coordinately controlled by a single controller, and the direction in which a temperature gradient is formed is controlled by selecting which temperature regulating elements are joined in a pair.

4. A thermal cycling instrument according to claim 2, wherein the angle between the first direction and the second direction is approximately 90 degrees.

5. A thermal cycling instrument according to claim 2, wherein the block comprises at least two temperature sensors, and wherein a first sensor is disposed near a first corner of said block, and a second sensor is disposed near a second corner diagonally opposite the first corner.

6. A thermal cycling instrument according to claim 1, wherein at least one of the temperature regulating elements comprises a thermoelectric heat pump.

7. A method for optimizing a thermal cycling program, which comprises the steps of:
a) programming said thermal cycling instrument to achieve a first thermal gradient of at least 2 degrees C. in a first direction during a first step;
b) programming said thermal cycling instrument to achieve a second thermal gradient of at least 2 degrees C. in a second direction during a second step, said second direction being at least 30 degrees and no more than 150 degrees different from said first direction;
c) placing at least three samples to be thermally cycled in thermal communication with said thermal cycling instrument, said samples arranged so that at least one pair of samples achieves different temperatures during the first step and a second pair of samples achieves different temperatures during the second step;
d) causing said thermal cycling instrument to thermally cycle said samples so that the first step and the second step are each repeated at least twice; and
e) assaying said samples for one or more quantifiable parameters to determine the optimum temperature in said thermal cycling program.

8. A method for optimizing a thermal cycling program for use in performing a biochemical reaction, which comprises the steps of:
a) selecting a biochemical reaction which comprises at least a first phase taking place substantially in a first temperature range and a second phase taking place substantially in a second temperature range, and a substantially inactive phase in a third temperature range intermediate between said first temperature range and said second temperature range;
b) programming a thermal cycling instrument to achieve a first step comprising a first programmed temperature within said first temperature range for a first programmed time;
c) programming said thermal cycling to achieve a temperature gradient step comprising a temperature gradient of at least 2 degrees C. in a defined direction such that at least one sample is held in the first or second temperature range, and at least one sample is held in the third temperature range for a third programmed time;
d) programming said thermal cycling instrument to achieve second step comprising a second programmed temperature within said second temperature range for a second programmed time;
e) placing a plurality of samples in thermal communication with a thermal cycling instrument arrayed in said defined direction;
f) causing said thermal cycling instrument to repeat at least twice a set of steps comprising the first step, the temperature gradient step, and the second step in sequence;
g) assaying said samples according to one or more quantifiable parameters to determine the optimum times for said first programming step or said programming second step.
